(19) United States
(12) Patent Application Publication
(10) Pub. No.: US 2026/0337367 A1
(43) Pub. Date: Oct. 29, 2026

(54) BIS(2-HYDROXYETHYL) TEREPHTHALATE SOLID EXHIBITING A PARTICULAR CRYSTALLINE FORM

(71) Applicants: IFP ENERGIES NOUVELLES, Rueil-Mailmaison (FR); JEPLAN, INC., Kawasaki City, Kanagawa (JP)

(72) Inventors: Guillaume BLANCKE, Rueil-Mailmaison Cedex (FR); David CHICHE, Rueil-Mailmaison Cedex (FR); Frederic FAVRE, Rueil-Mailmaison Cedex (FR); Damien LEINEKUGEL LE COCQ, Rueil-Mailmaison Cedex (FR)

(73) Assignees: IFP ENERGIES NOUVELLES, Rueil-Mailmaison (FR); JEPLAN, INC., Kawasaki City, Kanagawa (JP)

(21) Appl. No.: 19/122,420

(22) PCT Filed: Oct. 16, 2023
(86) PCT No.: PCT/EP2023/078640

§ 371 Date: Apr. 18, 2025

(30) Foreign Application Priority Data
Oct. 20, 2022 (FR) FR2210881

(51) Int. Cl. C08J 11/24; C07C 69/82; C08G 63/183

(52) U.S. Cl. CPC C08J 11/24; C07C 69/82; C08G 63/183; C08J 2367/02

(57) ABSTRACT

The present invention relates to a solid material predominantly composed of BHET having a crystalline form exhibiting a particular X-ray diffraction diagram, to its process of preparation, to a composition comprising said material and to the use of said composition for preparing a polyester.

### TECHNICAL FIELD

The invention relates to a solid material predominantly composed of bis(2-hydroxyethyl) terephthalate (BHET), exhibiting a novel crystalline form. This crystalline form makes it possible to advantageously obtain a needle morphology of the solid material, thus facilitating its drying. The invention also relates to a composition comprising said solid material and to the use of this composition for producing a terephthalate polyester, for example a polyethylene terephthalate (PET).

### PRIOR ART

Bis(2-hydroxyethyl) terephthalate (BHET) is a monomer of terephthalate polyesters and in particular of polyethylene terephthalate (PET).

BHET can be obtained by direct esterification of terephthalic acid with ethylene glycol or transesterification between dimethyl terephthalate and ethylene glycol, methods conventionally corresponding to the first reaction stage of conventional PET production processes. BHET can also be obtained by depolymerization of polyester, in particular of polyethylene terephthalate (PET), in the presence of ethylene glycol. This is referred to as chemical recycling of polyester, in particular of PET, since polyester waste, in particular PET waste, undergoes a chemical treatment (depolymerization) in order to obtain a monomer compound which is subsequently reused to again produce polyester, in particular PET and more particularly r-PET.

For example, Patent Application FR 3 053 691 describes a process for the depolymerization of a polyester feedstock comprising in particular from 0.1% to 10% by weight of pigments, by glycolysis in the presence of ethylene glycol. A bis(2-hydroxyethyl) terephthalate (BHET) monomer effluent, obtained after particular separation and purification stages, can be fed to a polymerization stage for the purpose of producing PET. Patent JP3715812 describes the production of refined BHET from PET, it being possible for the BHET obtained to be used as starting material in a process for the production of plastics.

Although they disclose the polymerization of monomeric products, in particular of BHET, resulting from the depolymerization of PET by glycolysis, the documents cited do not, however, give any information on the quality of the intermediates resulting from the depolymerization of PET, nor on the difficulty of the stages of purification, in particular of washing and drying, of the intermediates based on BHET.

In point of fact, the drying of BHET crystals is known to be problematic. For example, Patent U.S. Pat. No. 3,668,235 explains that the drying times for solid BHET are lengthy, which impacts the quality of the BHET, in particular by the appearance of a colouration and by a tendency for the crystals to agglomerate.

In order to facilitate drying and to thus limit the degradation of the product, it is important to reduce the operating time of the drying. The residual moisture level of the cake on exiting from filtration constitutes a good indicator of the ease of drying of the cake since drying will become easier as the amount of water to be removed decreases.

A technique known to a person skilled in the art for lowering the water content of a cake of a solid material is the use of centrifugal driers for liquid/solid separation, which achieve residual moisture levels 2 to 3 times lower than a simple filtration, the residual moisture level corresponding to the equilibrium between the capillary forces and the centrifugal forces (cf. M. Robatel et al., Centrifugation: Generalites, Theories [Centrifugation: General Points and Theories], Techniques de l′Ingénieur, A5550 V1, 1989, 10-17).

Patent Application WO 2021/032826 indicates that the moisture level of the cake of BHET crystals resulting from a filtration is typically in the range 20-50% by weight. This document then proposes a granulation of the BHET crystals in order to facilitate the drying of the solid, relying on the porosity of the granules to promote the transfers of matter and of heat.

Moreover, it is known that a needle-type morphology for crystals of a solid material makes it possible to obtain performance qualities superior to other morphologies, for example platelet morphology, in terms of filterability (cf. D. Bourcier et al., “Influence of Particle Size and Shape Properties on Cake Resistance and Compressibility during Pressure Filtration”, Chemical Engineering Science, 2016, 144, 176-187).

Patent JP 5189266 confirms the effect of the needle-type morphology of BHET on the quality of BHET, and in particular on the residual solvent content of a solid BHET obtained after crystallization and solid/liquid separation. However, this document gives no information as regards the crystalline form of the BHET obtained.

The paper by Miyake (A. Miyake, “Polymorphism of Bis-β-Hydroxyethyl Terephthalate”, Bulletin of the Chemical Society of Japan, 1957, 30 (4), 361-363) discloses four crystalline forms of BHET: the alpha crystalline form, which appears to be the most stable, and the beta, gamma and delta forms. However, this paper gives no indication as regards their macroscopic morphology and their properties, in particular of filterability and/or ease of drying. According to the paper by Alvarez-Castillo (A. Alvarez-Castillo et al., “Studies on the Crystallization of Polyethylene Terphthalate Oligomer”, Journal of Materials Science Letters, 14, 1995, 139-141), it would appear that the alpha crystalline form of BHET can impart a needle-type morphology to BHET crystals.

The present invention is targeted at a high-quality BHET and in particular at BHET crystals exhibiting the lowest possible moisture and/or residual solvent content. Thus, the inventors have discovered, surprisingly, a new crystalline form of BHET which inevitably results in a needle morphology and thus in improved properties of filterability and of drying of BHET crystals.

### SUMMARY OF THE INVENTION

A subject-matter of the invention is a solid material predominantly composed of BHET having a crystalline form exhibiting an X-ray diffraction diagram with the following mean values of 20 and of relative intensities Iᵣₑₗ greater than or equal to 5%:

TABLE 1

| 2 theta (°) | Iᵣₑₗ |
|---|---|
| 10.85 | vw |
| 18.35 | vw |
| 19.17 | vs |
| 21.76 | vw |
| 29.19 | w |
| 29.62 | vw |
| 38.82 | vw |
| 40.34 | vw |
| 49.18 | vw |

where vs=very strong; s=strong; m=medium; mw=medium weak; w=weak; vw=very weak. The relative intensity Iᵣₑₗ is given in relation to a relative intensity scale where a value of 100 is assigned to the most intense line of the X-ray diffraction diagram: vw<15; 15≤w<30; 30≤mw<50; 50≤m<65; 65≤s<85; vs≥85.

The advantage of the present invention lies in the fact that the particular crystalline form facilitates the filterability and the drying of BHET. This is because the solid material according to the invention exhibits a reduced residual moisture level on exiting from filtration, compared with the other crystalline forms, and is thus easier to dry. Moreover, the crystalline form of the solid BHET material according to the invention has a repeating structure which induces a needle morphology of the crystals which makes possible better filterability and improved washing of the crystals.

Moreover, another advantage of the present invention lies in the fact that the particular crystalline form of BHET is stable, in particular thermally stable.

Another advantage of the present invention lies in the origin of the solid BHET material and of the compositions which comprise it, since it can be obtained both by direct synthesis of BHET from terephthalic acid or dimethyl terephthalate and ethylene glycol but also originate advantageously from plastic recycling circuits, put in place in recent years by national and international organizations to fight plastic pollution. This is because the solid BHET material of the present invention and the composition which contains it can very advantageously be obtained on conclusion of processes for the depolymerization by glycolysis of polyester, such as PET, in the presence of diol, comprising purification stages, in particular a stage of crystallization of BHET. The BHET resulting from these depolymerization processes is then referred to as r-BHET and the PET prepared by polymerization from r-BHET is referred to as r-PET (as opposed to virgin resin or PET resulting from the direct polymerization of fresh ethylene glycol and terephthalic acid). Thus, the present invention contributes to the fight against plastic pollution.

The present invention thus also relates to the use of the composition comprising the solid BHET material for preparing a polyester, preferably a PET.

### LIST OF THE FIGURES

FIG. 1 represents an image of the solid A of Example 1, observed by optical microscopy.

FIG. 2 represents an image of the solid B of Example 1, observed by optical microscopy.

FIG. 3 represents the XRD diagram obtained for the solid A of Example 1.

FIG. 4 represents an XRD diagram of the solid B of Example 1.

FIG. 5 represents an XRD diagram of the solid C of Example 1.

FIG. 6 represents an XRD diagram of the solid D of Example 1.

### DESCRIPTION OF THE EMBODIMENTS

According to the invention, the terms “bis(2-hydroxyethyl) terephthalate” and “BHET” denote the same compound and are interchangeable. Likewise, the terms “bis(2-hydroxyethyl) isophthalate” and “BHEI” denote the same compound and are interchangeable. The terms “2-(2-hydroxyethoxy)ethyl 2-hydroxyethyl terephthalate” and “deg-BHET” denote the same compound and are themselves also interchangeable.

According to the invention, the term “polyester” denotes an advantageously saturated thermoplastic polymer (as opposed to thermosetting polyesters) having, as basic repeat units, diol diesters, and more particularly at least alkylene terephthalate units. The polymer chain can also comprise alkylene isophthalates and/or dialkyl terephthalate units. Thus, according to the invention, the term “polyester” is used to denote a poly(alkylene terephthalate) (or polyalkylene terephthalate). The polyester according to the invention can, for example, be poly(ethylene terephthalate) (or polyethylene terephthalate, PET), poly(butylene terephthalate) (or polybutylene terephthalate, PBT) or poly(trimethylene terephthalate) (or polytrimethylene terephthalate, PTT). The polyester according to the invention can also comprise, on its main polymer chain, other units, such as vinyl or polyol units, depending on the final properties desired for the polymer and depending on the targeted applications. According to the invention, the preferred polyester is polyethylene terephthalate or poly(ethylene terephthalate), also referred to simply as PET.

According to the invention, the terms “diol” and “glycol” are used without distinction and correspond to compounds comprising 2 hydroxyl-OH groups and preferably comprising between 2 and 12 carbon atoms, preferentially between 2 and 4 carbon atoms. The preferred diol is ethylene glycol, also referred to as monoethylene glycol or MEG.

Crystals are solids in which the atoms, ions or molecules are arranged in a three-dimensional space by repeating a structure periodically. The crystalline form corresponds to the description of this repeat structure. A solid can exist in different crystalline forms: this is then referred to as polymorphism. In a crystallization process, the production of one form rather than another is directed by the choice of the solvent and/or by the way the crystallization process is carried out. Each form is generally characterized by X-ray diffractometry (XRD). The group of peaks of a diffractogram obtained by XRD, in particular their positions and also preferably their intensities, characterizes the crystalline form. In the case of BHET, four crystal forms are known: alpha, beta, gamma and delta forms (cf. A. Miyake, “Polymorphism of Bis-β-Hydroxyethyl Terephthalate”, Bulletin of the Chemical Society of Japan, 1957, 30 (4), 361-363).

According to the present invention, the expressions “of between . . . and . . . ” and “between . . . and . . . ” are equivalent and mean that the limiting values of the interval are included in the range of values which are described. If such is not the case and if the limiting values are not included in the range described, such information will be provided by the present invention.

Within the meaning of the present invention, the various ranges of parameters for a given stage, such as the pressure ranges and the temperature ranges, can be used alone or in combination. For example, in the context of the present invention, a range of preferred pressure values can be combined with a range of more preferred temperature values.

Subsequently, particular embodiments of the invention may be described. It will be possible to carry them out separately or combined with one another, without limitation of combinations when this is technically feasible.

According to the invention, the pressures are absolute pressures and are given in MPa.

The invention thus relates to a solid material predominantly composed of BHET, preferably comprising BHET at a content by weight of greater than or equal to 50%, preferably of greater than or equal to 70%, preferentially of greater than or equal to 90%, very preferentially of greater than or equal to 95%, in a preferred way of greater than or equal to 98%, indeed even of greater than or equal to 99% (the percentages are relative to the total weight of the dry matter, that is to say without moisture or other solvent for example used during the process for the preparation of such solid and in particular during the crystallization stage, such as ethylene glycol or methanol or also a glycol ether), having a crystalline form exhibiting an X-ray diffraction diagram (or XRD diagram) with the following mean values of 20 and of relative intensities Iᵣₑₗ greater than or equal to 5%:

TABLE 1

| 2 theta (°) | Iᵣₑₗ |
|---|---|
| 10.85 | vw |
| 18.35 | vw |
| 19.17 | vs |
| 21.76 | vw |
| 29.19 | w |
| 29.62 | vw |
| 38.82 | vw |
| 40.34 | vw |
| 49.18 | vw |

where vs=very strong; s=strong; m=medium; mw=medium weak; w=weak; vw=very weak. The relative intensity Iᵣₑₗ is given in relation to a relative intensity scale where a value of 100 is assigned to the most intense line of the X-ray diffraction diagram: vw<15; 15≤w<30; 30≤mw<50; 50≤m<65; 65≤s<85; vs≥85.

The solid material predominantly composed of BHET according to the invention can also be referred to as BHET material or solid BHET, in the continuation of this description.

According to a particular embodiment of the invention, the solid material, which is predominantly composed of BHET having the crystalline form for which the XRD diagram is presented in Table 1, exhibits a single crystalline form. In other words, it exhibits only the crystalline form exhibiting the X-ray diffraction diagram for which the mean values of 20 and of relative intensities are given in Table 1. Preferably, the BHET material which exhibits a single crystalline form, that for which the XRD diagram is presented in Table 1, does not exhibit an amorphous form.

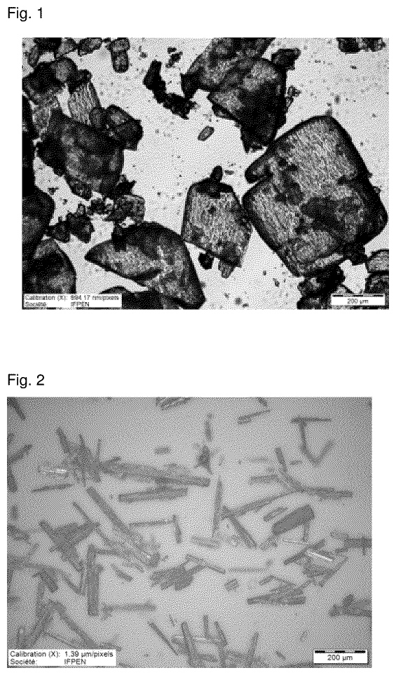

According to another particular embodiment of the invention, the solid material is predominantly composed of BHET which exhibits the crystalline form exhibiting the X-ray diffraction diagram represented by the values of 20 and of relative intensities greater than or equal to 5% of Table 1 and of another crystalline form of BHET preferably chosen from the alpha form, the beta form, the delta form or the gamma form of BHET and a combination of at least two of these crystalline forms. The XRD diagrams of the alpha, beta, delta and gamma forms of BHET are represented in FIG. 1 and were determined by the Miyake group (A. Miyake, “Polymorphism of Bis-β-Hydroxyethyl Terephthalate”, Bulletin of the Chemical Society of Japan, 1957, 30 (4), 361-363). According to this particular embodiment of the invention, the BHET material can also exhibit an amorphous form. Preferably, the BHET material of this particular embodiment does not comprise an amorphous form.

According to the invention, the X-ray diffraction (XRD) analysis carried out on the BHET material makes it possible to confirm the presence of the crystalline form(s) of BHET. According to the invention, the solid BHET material exhibits the X-ray diffraction diagram including at least the lines listed in Table 1. Preferably, the X-ray diffraction diagram does not contain other lines of significant intensity (that is to say, of intensity greater than or equal to 5% of the intensity of the most intense line of the XRD diagram) than those listed in Table 1. For a person skilled in the art, the essential characteristic on a XRD diagram is the position of the peaks (values of 2 theta); the relative intensities are often given for information purposes.

The X-ray diffraction diagram (or XRD diagram) is obtained by radiocrystallographic analysis by means of a diffractometer using the conventional powder method with copper Kα₁ (λ=1.5406 Å) radiation. The position of the diffraction peaks (or lines) is represented by the angle 2θ. An absolute error Δ (2θ), assigned to the 2θ measurement, equal to ±0.1° is commonly accepted. The relative intensity Iᵣₑₗ assigned to each value of dₕₖₗ is measured according to the height of the corresponding diffraction peak (or line). The X-ray diffraction diagram of the solid material predominantly comprising BHET according to the invention comprises at least the lines given in Table 1.

Very advantageously, the solid material according to the invention is in the form of needles.

The present invention also relates to a composition comprising the BHET material according to the invention. Preferably, the composition comprising the BHET material according to the invention is in solid form or in liquid form (that is to say, a composition macroscopically in liquid form, for example a suspension or a slurry being compositions in liquid form) and more particularly in solid form, in the form of a slurry or in the form of a suspension of solid particles of the BHET material according to the invention in suspension in a solvent. The composition comprising the BHET material according to the invention can thus be in solid or liquid (suspension or slurry) form and additionally comprise a solvent, preferably chosen from an aqueous solvent, in particular water, an alcoholic solvent, for example methanol or a diol, such as ethylene glycol, or a solvent composed of a glycol mono- or diether; preferably, the solvent is water.

According to a particular embodiment of the invention, the composition comprising the BHET material according to the invention is a solid composition and additionally comprises a solvent, preferably at a content of less than or equal to 20% by weight, preferably of less than or equal to 15% by weight, preferentially of less than or equal to 10.0% by weight and more particularly of less than or equal to 5.0% by weight, indeed even of less than or equal to 1.0% by weight.

According to another particular embodiment of the invention, the composition comprising the BHET material according to the invention is a composition in liquid form, more particularly a composition of slurry or suspension type, which comprises a solvent and solid particles of the BHET material according to the invention, preferably between 1% and 75% by weight, preferentially between 5% and 45% by weight, in a preferred way between 15% and 35% by weight of solid material according to the invention, with respect to the total weight of said composition.

Advantageously, the composition according to the invention is capable of being obtained by, preferably is obtained by, a process for the treatment of a polyester feedstock, preferably comprising PET, which comprises a stage of depolymerization of the polyester feedstock, in particular of the PET which it contains, and preferably followed by at least one separation-purification stage. The depolymerization stage can employ a depolymerization by glycolysis in the presence of ethylene glycol or by methanolysis in the presence of methanol. In the latter case, an additional stage of transesterification in the presence of ethylene glycol is then necessary. Preferably, the depolymerization stage employs a depolymerization by glycolysis in the presence of ethylene glycol. The process for the treatment of a polyester feedstock, preferably comprising PET, can for example comprise, as stage of purification of the effluent obtained by depolymerization of the polyester feedstock, in particular a stage of crystallization of the BHET from water, from ethylene glycol or from a glycol mono- or diether, preferably from water.

According to a particular embodiment of the invention, the composition according to the invention may additionally comprise bis(2-hydroxyethyl) isophthalate (BHEI), preferably in a molar amount such that the (BHEI/[BHET+BHEI]) molar ratio of the number of moles of BHEI with respect to the number of moles of the combination of BHET and BHEI which are present in the composition is less than or equal to 10.0 mol %, preferably less than or equal to 5.0 mol %, preferentially less than or equal to 1.0 mol % and in a preferred way less than or equal to 0.5 mol %. In addition, if the composition comprises BHEI, the (BHEI/[BHET+BHEI]) molar ratio is greater than or equal to 0.001 mol %, preferably greater than or equal to 0.01 mol %, preferentially greater than or equal to 0.05 mol %.

According to another particular embodiment of the invention, the composition based on BHET according to the invention may additionally comprise 2-(2-hydroxyethoxy)ethyl 2-hydroxyethyl terephthalate (deg-BHET), preferably in a molar amount such as the (deg-BHET/[BHET+deg-BHET]) molar ratio of the number of moles of deg-BHET with respect to the number of moles of the combination of BHET and deg-BHET which are present in the composition is less than or equal to 10.0 mol %, preferably less than or equal to 5.0 mol %, preferentially less than or equal to 1.0 mol %. In addition, if the composition comprises deg-BHET, the (deg-BHET/[BHET+deg-BHET]) molar ratio is greater than or equal to 0.001 mol %, preferably greater than or equal to 0.05 mol %, preferentially greater than or equal to 0.10 mol %, in a preferred way greater than or equal to 0.50 mol %.

One or the other or these two particular embodiments of the invention may possibly be found to be the case(s) for products obtained on conclusion of processes for the treatment of polyester feedstocks, which comprise a depolymerization stage.

The present invention thus relates to a process for the preparation of a composition according to the invention comprising:
a stage of depolymerization of a polyester feedstock, preferably comprising PET, employing a depolymerization by glycolysis in the presence of ethylene glycol or by methanolysis in the presence of methanol, preferably a depolymerization by glycolysis in the presence of ethylene glycol; then
at least one purification stage, preferably comprising a stage of crystallization from water, from ethylene glycol or from a glycol mono- or diether, preferably from water.

Very advantageously, the preparation process comprises, consists of, the depolymerization process described in Patent FR 3 053 691, the decolouration stage of which comprises an adsorption stage, and can additionally comprise a stage of purification by crystallization of the BHET from water, from ethylene glycol or from a glycol mono- or diether, preferably from water.

The BHET material according to the invention, which exhibits the particular crystalline form for which the XRD diagram is shown in Table 1, advantageously makes possible the filtration and the drying of the composition according to the invention which contains it and which is obtained on conclusion of such a preparation process. As these filtration and drying stages are facilitated, the solid according to the invention, obtained on conclusion of such stages, thus advantageously comprises a reduced residual solvent content, in particular a reduced residual moisture content, which makes it possible to use it in a polymerization stage, without an additional treatment that is costly in terms of energy.

The composition which comprises the BHET material according to the invention very advantageously makes it possible to obtain, after polymerization, a polyester, preferably a PET, and in particular an r-PET, exhibiting a light, indeed even colourless, colouration.

Thus, the invention also relates to the use of the composition according to the invention, optionally mixed with at least one dicarboxylic acid, preferably chosen from terephthalic acid and isophthalic acid, and/or at least one diol, preferably chosen from ethylene glycol, diethylene glycol, butylene glycol, cyclohexanedimethanol, neopentyl glycol or their mixtures, the preferred diol being ethylene glycol, in order to prepare a polyester, preferably a PET.

The invention thus also relates to a process for the production of a polyester comprising, preferably consisting of:
a) a stage of esterification of a feedstock comprising at least the composition according to the invention, and optionally at least one dicarboxylic acid, preferably chosen from terephthalic acid and isophthalic acid, and/or at least one diol, preferably chosen from ethylene glycol, diethylene glycol, butylene glycol, cyclohexanedimethanol, neopentyl glycol or their mixtures, the preferred diol being ethylene glycol, then
b) a polycondensation stage.

Advantageously, stage a) is carried out at a temperature of between 150 and 350 °C, preferably between 200 and 300 °C, in a preferred way between 250 and 285 °C. Preferably, stage a) is carried out at a pressure between 0.05 and 1.0 MPa, preferably between 0.1 and 0.5 MPa. Very advantageously, stage a) is carried out with a residence time between 0.5 and 10.0 hours, preferably between 1.0 and 6.0 hours, the residence time being defined here as the ratio of the reaction volume of a reactor employed in stage a) to the flow rate by volume of the liquid stream exiting from said reactor.

A polymerization catalyst, preferably based on antimony, titanium, germanium, aluminium, zinc acetate, calcium acetate and/or manganese acetate, can optionally be introduced in stage a).

The reaction carried out in stage a) generates a diol compound which is advantageously separated during stage a), for example by withdrawal, distillation and/or adsorption. Water can also be formed. The water then formed is itself also advantageously separated during stage a).

Advantageously, the process for the production of a polyester according to the invention comprises a polycondensation stage b) on conclusion of stage a). Stage b) can advantageously employ one or more, preferably one or two, polycondensation substage(s), for example at least one, preferably one, liquid or melt phase polycondensation substage, optionally followed by at least one, preferably one, solid phase polycondensation substage.

Very advantageously, the polycondensation stage b) employs at least one polymerization section, preferably one or two polymerization sections, advantageously operated in the liquid or melt phase, said polymerization section(s) being employed at a temperature greater than the temperature at which stage a) is carried out, preferably at a temperature of between 19° and 400 °C, preferentially between 220 and 350 °C, in a preferred way between 265 and 300 °C, preferably at a pressure between 0.01 and 100.00 kPa, preferentially between 0.05 and 10.00 kPa, and in a preferred way with a residence time of between 0.1 and 5.0 hours, preferably between 0.5 and 4 hours, preferentially between 1.0 and 3.0 hours. The residence time in the polymerization section of stage b) is defined as the ratio of the reaction volume of a reactor employed in said polymerization section to the flow rate by volume of the liquid stream, comprising the polyester produced, exiting from said reactor.

The polymerization reaction can optionally be continued in a polycondensation section located downstream of the polymerization section and operated in the solid phase, preferably at a temperature (in particular a product temperature) of between 190 and 250 °C, preferentially between 200 and 230 °C, depending on whether this operation is carried out in continuous mode or in batch mode. The polycondensation section can preferably be operated under an inert atmosphere, for example under a stream of nitrogen, at a pressure close to atmospheric pressure, or under vacuum (in particular at a pressure between 0.01 and 100 kPa, indeed even between 0.01 and 10 kPa). The residence time (defined as the time during which the product is subjected to the polycondensation conditions in said polycondensation section) is of between 5 and 20 hours, preferably between 10 and 16 hours. Said polycondensation section can advantageously be preceded by a crystallization section, thus located between the polymerization section and the polycondensation section, in which the polyester formed, obtained at the end of the polymerization section, is advantageously crystallized, it being possible for said crystallization section to be operated at a temperature preferably between 110 and 210 °C and for a residence time (defined as the time during which the product is subjected to the crystallization conditions in said section) preferably between 0.5 and 6 hours.

Stage b) is preferably carried out in the presence of a polymerization catalyst, in particular based on antimony, titanium, germanium, aluminium, zinc acetate, calcium acetate and/or manganese acetate.

Additives can be introduced in the polycondensation stage b). The additives optionally introduced in stage b) can, for example, be: agents for inhibiting etherification side reactions, such as, for example, amines (n-butylamine, diisopropylamine or triethylamine), sodium hydroxide or organic hydroxides or lithium carbonate, stabilizing agents, such as phosphites or phosphates, and compounds of polyamide type for reducing the amount of degradation product, such as acetaldehyde.

The following Figures and Examples illustrate the invention without limiting the scope thereof.

### EXAMPLES

### Example 1: Solids

Two solids, solid A and solid B, obtained on conclusion of processes for the depolymerization by glycolysis of PET waste and purification by crystallization from water (gradual lowering of the temperature from 60 °C to 20 °C), are recovered after filtration. The solids A and B recovered comprise at least 98.5% by weight of BHET with respect to their dry solid weight. A fraction of the solid B is subsequently dried in an oven at 30 °C for 15 hours in order to obtain a solid C. A fraction of the solid C is subsequently placed at 60 °C for 15 hours in order to obtain the solid D.

The solids A and B were observed by optical microscopy. The photos of these observations are shown in FIGS. 1 and 2 respectively for the solids A and B.

The solid A has a platelet-type morphology (FIG. 1).

The solid B has a needle-type morphology (FIG. 2).

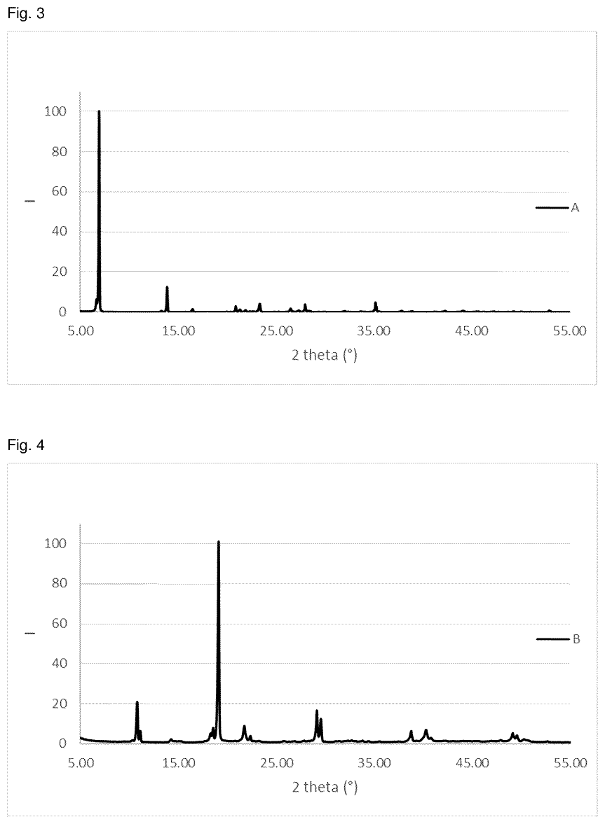

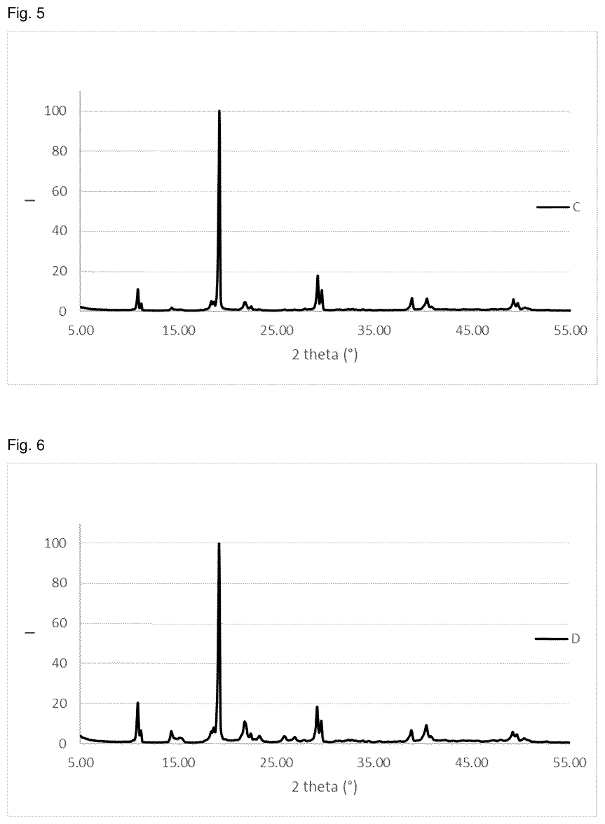

The XRD diagrams of the solids A, B, C and D are determined by radiocrystallographic analysis by means of a diffractometer using the conventional powder method with copper Kα₁ (λ=1.5406 Å) radiation. The position of the diffraction peaks (or lines) is represented by the angle 2θ measured with an absolute error Δ(2θ) equal to ±0.1°. The relative intensity Iᵣₑₗ is measured from the height of the corresponding diffraction peak (or line). The XRD diagrams of the solids A, B, C and D are shown in FIGS. 3, 4, 5 and 6, respectively, and presented in Table 2 below.

TABLE 2

| Solid A | | Solid B | | Solid C | | Solid D | |
|---|---|---|---|---|---|---|---|
| 2 theta (°) | Iᵣₑₗ | 2 theta (°) | Iᵣₑₗ | 2 theta (°) | Iᵣₑₗ | 2 theta (°) | Iᵣₑₗ |
| 6.65 | vw | 10.80 | w | 10.87 | vw | 10.90 | w |
| 6.92 | vs | 11.13 | vw | | | 11.20 | vw |
| 13.87 | vw | | | | | 14.31 | vw |
| 23.37 | vw | 18.29 | vw | 18.39 | vw | 18.39 | vw |
| 35.16 | vw | 18.55 | vw | | | 18.62 | vw |
| | | 19.12 | vs | 19.19 | vs | 19.19 | vs |
| | | 21.73 | vw | 21.79 | vw | 21.79 | vw |
| | | 29.15 | w | 29.21 | w | 29.21 | s |
| | | 29.55 | vw | 29.65 | vw | 29.65 | vw |
| | | 38.77 | vw | 38.84 | vw | 38.84 | vw |
| | | 40.28 | vw | 40.38 | vw | 40.38 | vw |
| | | 49.13 | vw | 49.20 | vw | 49.20 | vw |

where vs=very strong; s=strong, m=medium; mw=medium weak; w=weak; vw=very weak. The relative intensity Iᵣₑₗ is given in relation to a relative intensity scale where a value of 100 is assigned to the most intense line of the X-ray diffraction diagram: vw<15; 15≤w<30; 30≤mw<50; 50≤m<65; 65≤s<85; vs≥85.

The solid A corresponds to the α form.

The solids B, C and D correspond to the crystalline form in accordance with the invention.

From the XRD diagrams, it appears that the crystalline form according to the invention is stable since the XRD diagrams of the solids C (drying of the solid at 30 °C for 15 hours) and D (drying of the solid at 30 °C for 15 hours and then at 60 °C for 15 hours) are not or only slightly modified with respect to that of the solid B.

### Example 2: Centrifugal Spinning of the Solids E (not in Accordance) and F (in Accordance with the Invention)

The solids E and F, obtained by crystallization of solutions of BHET in water by gradual reduction of the temperature from 60 °C to 20 °C respectively for 4 hours and 6 hours, and recovered after filtration, comprise at least 98.5% by weight of BHET with respect to their dry solid weight. They are observed under an optical microscope and their XRD diagram is determined, according to the same method as that described in detail in Example 1.

The solid E has a platelet-type morphology and exhibits an a crystallographic form.

The solid F has a needle-type morphology and exhibits a crystallographic form in accordance with the invention.

Each of the solids E and F undergoes washing with water such that each mixture is a suspension of 20% by weight of solid in 80% by weight of water, then a solid-liquid separation by centrifugal spinning at 20 °C.

The residual water content of each solid after washing is determined by the loss in weight of the solids after drying in an oven at 40 °C under vacuum for 15 hours.

The results obtained for the two solids E and F are presented in Table 3.

TABLE 3

| | Solid E | Solid F |
|---|---|---|
| BHET content (%) | 98.5 | 98.5 |
| Crystalline form | α | according to the |
| | | invention |
| Residual moisture content | 26 | 10 |
| after centrifugal spinning | | |
| (%) | | |

The results show that the solid F with a crystalline form according to the invention and exhibiting a needle-type form makes it possible to achieve a residual moisture content (10%) which is substantially lower than that achieved by the solid E having an a crystalline form and exhibiting a platelet-type form. The solid F will thus be much easier to dry compared with the solid F because there is less water to be removed.

1. Solid material predominantly composed of BHET having a crystalline form exhibiting an X-ray diffraction diagram with the following mean values of 2θ and of relative intensities Iᵣₑₗ greater than or equal to 5%:
TABLE 1

| 2 theta (°) | Iᵣₑₗ |
|---|---|
| 10.85 | vw |
| 18.35 | vw |
| 19.17 | vs |
| 21.76 | vw |
| 29.19 | w |
| 29.62 | vw |
| 38.82 | vw |
| 40.34 | vw |
| 49.18 | vw |

where vs=very strong; s=strong; m=medium; mw=medium weak; w=weak; vw=very weak. The relative intensity Iᵣₑₗ is given in relation to a relative intensity scale where a value of 100 is assigned to the most intense line of the X-ray diffraction diagram: vw<15; 15≤w<30; 30≤mw<50; 50≤m<65; 65≤s<85; vs≥85.

2. Material according to claim 1, exhibiting a single crystalline form.

3. Material according to claim 1, having the crystalline form exhibiting the X-ray diffraction diagram represented by the values of 20 and of relative intensities of Table 1 and another crystalline form, preferably chosen from the alpha form, the beta form, the delta form or the gamma form of BHET and a combination of at least two of these crystalline forms.

4. Material according to claim 1, comprising BHET having said crystalline form, in a content by weight of greater than or equal to 90%, preferably of greater than or equal to 95%, in a preferred way of greater than or equal to 98%, the percentages being given with respect to the total weight of the dry material.

5. Material according to claim 1, in the form of needles.

6. Solid or liquid composition comprising the material according to claim 1.

7. Composition according to claim 6, additionally comprising a solvent, preferably an aqueous solvent, an alcoholic solvent, for example methanol, or a diol, or also a glycol mono- or diether, the preferred solvent being water.

8. Solid composition according to claim 6, additionally comprising a solvent, and preferably at a content by weight of less than or equal to 20% by weight, preferably of less than or equal to 15% by weight.

9. Composition according to claim 6, capable of being obtained by a process for the treatment of a polyester feedstock, preferably comprising PET, which comprises a stage of depolymerization by glycolysis in the presence of ethylene glycol, and preferably followed by at least one purification and/or separation stage, for example comprising a stage of crystallization of BHET, in particular from water.

10. Process for the preparation of a composition according to claim 6, comprising:
a stage of depolymerization of a polyester feedstock, preferably comprising PET, employing a depolymerization by glycolysis in the presence of ethylene glycol or by methanolysis in the presence of methanol, preferably a depolymerization by glycolysis in the presence of ethylene glycol; then
at least one purification stage, preferably comprising a stage of crystallization from water, from ethylene glycol or from a glycol mono- or diether, preferably from water.

11. A method for preparing a polyester, preferably a PET, comprising preparing said polyester from the composition according to claim 6.
